(19) United States
(12) Patent Application Publication
(10) Pub. No.: US 2003/0198654 A1
(43) Pub. Date: Oct. 23, 2003

(54) COSMETIC FORMULATION THAT PROVIDES FOR A SUSPENSION OF BEADS

(76) Inventor: Christopher L. Palazzolo, O'Fallon, MO (US)

Correspondence Address: BLACKWELL SANDERS PEPER MARTIN LLP, 720 OLIVE STREET, SUITE 2400, ST. LOUIS, MO, 63101, US

(21) Appl. No.: 10/249,409

(22) Filed: Apr. 7, 2003

Related U.S. Application Data
(60) Provisional application No. 60/319,197, filed on Apr. 22, 2002.

(51) Int. Cl. A61K 7/06; A61K 7/11; A61K 7/075; A61K 7/08; A61K 9/14

(52) U.S. Cl. 424/401; 424/70.13; 424/70.21; 424/70.24

(57) ABSTRACT

A cosmetic composition that allows for the suspension of beads of differing sizes is disclosed. This cosmetic composition will allow the beads to remain suspended but is not too viscous to destroy or significantly reduce the desired qualities in the cosmetic composition, e.g., body gel. This includes the primary ingredients of a carbomer solution (with a neutralizing agent) and a gum solution. Optional ingredients can include, but are not limited to, a surfactant, a chemical to lower the freezing point, a preservative, a fragrance, and hair and skin treatment additives. The beads are preferably formed from a wide variety of shell materials that house a core oil. The aesthetic qualities of this cosmetic composition are very pleasing in addition to the performance provided by the suspended beads.

### CROSS REFERENCE TO RELATED APPLICATIONS

This patent application claims U.S. Provisional Patent Application Serial No. 60/319,197 filed Apr. 22, 2002.

### BACKGROUND OF INVENTION

Although particles have been used in cosmetic products, there is a significant problem with being able to keep particles of various sizes suspended in a cosmetic composition. Typically, the larger particles tend to drop to the bottom of the container within a few days. Examples of cosmetic compositions include: body gels; lotions; creams; perfumes; moisturizers; and so forth. Particles that have a contrasting color in relation to the cosmetic composition provide a very aesthetically pleasing look to the product. Another problem is that in order to create the type of cosmetic composition that allows the larger particles to remain suspended, the cosmetic composition is usually so viscous that performance of the product is significantly impaired.

An example includes that disclosed in U.S. Pat. No. 6,251,409, which issued to Hegyi et al. on Jun. 26, 2001, which is directed to using translucent or transparent particles in a cosmetic base, but completely fails to address how to keep the particles of various sizes suspended while maintaining product functionality with a reasonable viscosity. Other examples of suspending beads with gums have been utilized by Lipo Technologies, Inc., however, beads of the same size would be required and the viscosity needed for the larger beads will negate the effectiveness and value of the cosmetic composition for the consumer.

The present invention is directed to overcoming one or more of the problems set forth above by providing a novel composition and a process for making a composition which contains a carbomer solution and a gum solution. As used herein the term “composition” and “formulation” are synonymous. Carbomers are high molecular weight network polymers with acrylic acid backbones and small amounts of polyether crosslinking agents as described in “Advances in Carbomer Polymer Technology”, Desai et al, Cosmetics and Toiletries Manufacture Worldwide, 1998.

### SUMMARY OF INVENTION

A cosmetic composition that provides for a suspension of beads is disclosed. The preferred cosmetic composition is a body gel; however, the present invention can be extended to a wide variety of other cosmetic compositions. This cosmetic composition includes a carbomer solution or pre-mix and a gum solution or pre-mix. Preferably, the beads include a shell material that encloses an oil. Illustrative, but nonlimiting, examples of optional ingredients could include a surfactant, a chemical to lower the freezing point, a preservative, a fragrance and a hair and/or skin treatment additive.

The carbomer solution or pre-mix preferably includes a carbomer in a range from about 0.01% by weight to about 10.0% by weight, water in a range from about 88.0% by weight to about 99.98% by weight and a neutralizing agent in a range from about 0.01% by weight to about 2.0% by weight of the carbomer solution or pre-mix. Illustrative, but nonlimiting, examples of carbomers include SF-1 (AQUA™) carbomer, carbomer 940, carbomer Ultrez 10™, carbomer 1342 and carbomer ETD 2020™. Illustrative, but nonlimiting, examples of neutralizing agents include triethanolamine (TEA), sodium hydroxide, tetrahydroxypropyl dimethylamine (TE) and diisopropylamine (DIPA). The above-described carbomers are manufactured by Noveon, Inc. of Cleveland, Ohio.

The gum solution or pre-mix preferably includes a gum in a range from about 0.01% by weight to about 10.0% by weight and water in a range from about 90.0% by weight to about 99.99% by weight of the gum solution or pre-mix. Illustrative, but nonlimiting, examples of gum-like materials include carboxymethylcellulose, hydroxymethylcellulose, hydroxyethylcellulose, magnesium aluminum silicate, xanthan gum and guar gum.

The surfactant is preferably in a range from about 1.0% by weight to about 75.0% by weight of the cosmetic composition with the preferred surfactant being triethanolamine (TEA) lauryl sulfate, however, other illustrative, but nonlimiting, examples of surfactants include sodium lauryl sulfate, sodium laureth sulfate, EnvAddressee1 ammonium EnvAddressee1 lauryl sulfate, ammonium laureth sulfate, sodium C14-16 olefin sulfonate, sodium xylene sulfonate, decyl polyglucoside, cocamide DEA, cocamide MEA, lauramide DEA, lauramide MEA, sodium cocoamphodiacetate, cocoamidopropyl betaine, cocoamide oxide, cocoamidopropyl hydroxysultaine and disodium laureth sulfosuccinate or any combination thereof. Preferably, the surfactant is present in the cosmetic composition at about 15-40% by weight of said composition.

The chemical to lower the freezing point, if added, is preferably in a range from about 0.01% by weight to about 5.0% by weight of the cosmetic composition and is preferably propylene glycol, however, other illustrative, but nonlimiting, examples of chemicals that can lower the freezing point of a cosmetic composition include diethylene glycol (DEG), glycerine, polysorbate-20 and polysorbate-80.

The preservative is preferably in a range from about 0.01% by weight to about 2.0% by weight of the cosmetic composition and is preferably dimethylol dimethyl hydantoin (DMDMH), however, other illustrative, but nonlimiting, examples of preservatives include methylparaben, propylparaben, methylchloroisothiazolinone and methylisothiazolinone.

Any of a wide variety of fragrances can be utilized with one illustrative, but nonlimiting example being FREE SPIRIT™ BELLEAIRE™ (manufactured by Belle-Aire Fragrances of Mundelein, Ill.) with a range from about 0.01% by weight to about 5.0% by weight or more of the cosmetic composition. The hair and/or skin treatment additive is preferably in a range from about 0.01% by weight to about 5.0% by weight of the cosmetic composition and is preferably panthenol, however, other illustrative, but nonlimiting, examples of hair and/or skin treatment additives include centrimonium chloride, glycereth-26, polyquaternium-7, polyquaternium-10, amodimethicone, cyclomethicone, dimethicone, dimethicone copolyol, phenyl trimethicone, stearamidopropyl dimethiamine, quaternium 15 and quaternium 18.

The beads include a shell housing, a core oil and can range from about 0.01% by weight to about 2.0% by weight of the cosmetic composition. Illustrative, but nonlimiting materials that can be utilized for the shell housing can include agar, gelatin, gelatin coacervate, methyolphenols, and polyoxymethylene urea. Illustrative, but nonlimiting examples of core oils can include both natural and synthetic oils such as aloe vera, avocado, boretsh, frankincense, grape seed, jojoba, lavender, macadamia nut, meadowfoam, rose hip, rosemary, safflower, seaweed, sesame, soybean, sunflower, sweet almond oil, tea tree, caprylic/capric triglyceride, cyclomethicone, isopropyl myristate, mineral oil, mineral oil substitute, polydimethyl siloxane, silicone oil, tridecyl trimellitate and neopentylglycol dicaprylate/dicapate. Other core oils can include vitamins such as Vitamin A (Retinyl Palmitate and Retinol), Vitamin B5 (Panthanol Triacetate), Vitamin C (ascorbic acid) and Vitamin E (Topopherol and Acetate). Moreover, core oils can include benzophenone, biobantical extracts, copper chlorophyll, menthol, and methylsalicylate. Moreover, a wide variety of fragrances and flavors in addition to full gamut of solids and other compounds that can also be utilized.

A process for suspending beads in a cosmetic composition is also disclosed. The process includes mixing a carbomer in a range from about 0.01% by weight to about 10.0% by weight with a neutralizing agent in a range from about 0.01% by weight to about 2.0% by weight with water in a range from about 88.0% by weight to about 99.98% by weight to create a carbomer solution or pre-mix, mixing a gum-like substance in a range from about 0.01% by weight to about 10.0% by weight with water in a range from about 90.0% weight to about 99.99% by weight to create a gum solution or pre-mix, mixing the carbomer solution or pre-mix with a surfactant that ranges from about 1.0% by weight to about 75.0% by weight of the cosmetic composition, adding the gum pre-mix, then adding optional ingredients, and then adding beads of shells that enclose core oils.

The optional ingredients include adding a chemical to lower the freezing point in a range from about 0.01% by weight to about 5.0% by weight of the cosmetic composition, adding a preservative that is in a range from about 0.01% by weight to about 2.0% by weight of the cosmetic composition, adding a fragrance that is in a range from about 0.01% by weight to about 5.0% by weight of the cosmetic composition and adding a hair and/or skin treatment additive(s) in a range from about 0.01% by weight to about 5.0% by weight of the cosmetic composition. The beads of shell material housing a core oil can range from about 0.01% by weight to about 2.0% by weight of the cosmetic composition and are mixed-in as the final step in the process.

Thus, the present invention contemplates a process for suspending beads in a cosmetic composition which comprises mixing a carbomer in a range from about 0.01% by weight to about 10.0% by weight with a neutralizing agent in a range from about 0.01% by weight to about 2.0% by weight to create a carbomer solution having a substantially uniform consistency; mixing a gum-like substance in a range from about 0.01% by weight to about 10.0% by weight to create a gum solution; mixing the carbomer solution with the gum solution until a cosmetic composition having a substantially uniform consistency is achieved; and adding to the cosmetic composition at least one bead having a shell that encloses a core oil. In one aspect of the invention the amount of carbomer solution present in the cosmetic composition is from about 10-90% by weight of the cosmetic composition. The amount of gum solution present in the cosmetic composition is from about 10-90% by weight of the cosmetic composition. Preferably, the amount of carbomer solution present in the cosmetic composition is from about 15-40% by weight of the cosmetic composition. The amount of gum solution present in the cosmetic composition is from about 15-40% by weight of the cosmetic composition. Most preferably, the amount of carbomer solution present in the cosmetic composition is from about 30-40% by weight of the cosmetic composition. The amount of gum solution present in the cosmetic composition is from about 30-40% by weight of the cosmetic composition.

These are merely illustrative aspects of the present invention and should not be deemed an all-inclusive listing of the innumerable aspects associated with the present invention. These and other aspects will become apparent to those skilled in the art in light of the following disclosure.

### DETAILED DESCRIPTION

The present invention is directed to a cosmetic composition and a process to provide a cosmetic composition that allows beads of various sizes to remain suspended in solution. The preferred type of cosmetic composition is a body gel composition, however, the concept can be extended to: lotions; creams; perfumes; moisturizers; and so forth.

There are two pre-mix solutions associated with the present invention where the first pre-mix is a carbomer solution or carbomer pre-mix. This carbomer solution or carbomer pre-mix preferably includes mixing a carbomer into an aqueous solution. The preferred type of carbomer is an SF-1 (AQUA™) carbomer, however, there can be numerous substitutions for this specific carbomer including carbomer 940, carbomer Ultrez 10™, carbomer 1342 and carbomer ETD 2020™. The percentage of carbomer in the carbomer solution or carbomer pre-mix can range from about 0.01% by weight to about 10.0% by weight with a preferred range from about 1.0% by weight to about 3.0% by weight with the optimal percentage of 2.0% by weight. Likewise, the percentage of water in carbomer solution can range from about 88% by weight to about 99.98% by weight with a preferred range from about 95.0% by weight to about 98.99% by weight with a optimal percentage of 97.5% by weight. In order to neutralize the pH, triethanolamine (TEA) is added as a neutralizing agent. The percentage of triethanolamine (TEA) in the carbomer solution or carbomer pre-mix can range from about 0.01% by weight to about 2.0% by weight with a preferred percentage of 0.5% by weight. Numerous other potential neutralizing agents can be utilized including sodium hydroxide, tetrahydroxypropyl dimethylamine (TE) and diisopropylamine (DIPA) as illustrative, but nonlimiting, examples.

The second pre-mix is a gum solution or gum pre-mix. This gum pre-mix preferably includes carboxymethylcellulose in an aqueous solution. This carboxymethylcellulose is sprinkled into the water with heavy agitation to form a thick, clear gel. The percentage of carboxymethylcellulose in the gum solution or pre-mix can range from about 0.01% by weight to about 10.0% by weight with a preferred range from about 1.0% by weight to about 3.0% by weight with the optimal percentage of 2.0% by weight while the percentage of water in the gum solution can range from about 99.99% by weight to about 90.0% by weight with a preferred range from about 99.0% by weight to about 97.0% by weight with the optimal percentage of 98.0% by weight. Numerous other gum-like materials can be utilized including hydroxymethylcellulose, hydroxyethylcellulose, magnesium aluminum silicate, xanthan gum and guar gum as illustrative, but nonlimiting, examples.

The first step in the composition process, after creating both the carbomer solution or carbomer pre-mix and the gum solution or gum pre-mix includes mixing a common surfactant, such as triethanolamine (TEA) lauryl sulfate in a vessel and then adding the previously described carbomer pre-mix. When the solution achieves a uniform consistency, the previously described gum pre-mix is added until uniformity is again achieved and then the batch of cosmetic composition is left standing. The percentage of triethanolamine (TEA) lauryl sulfate in solution can range from about 1.0% by weight to about 75.0% by weight with a preferred percentage of 25.0% by weight. Numerous other surfactant substitutions can be utilized instead of triethanolamine (TEA) lauryl sulfate including sodium lauryl sulfate, sodium laureth sulfate, ammonium lauryl sulfate, ammonium laureth sulfate, sodium C14-16™ olefin sulfonate, sodium xylene sulfonate, decyl polyglucoside, cocamide DEA, cocamide MEA, lauramide DEA, lauramide MEA, sodium cocoamphodiacetate, cocoamidopropyl betaine, cocoamide oxide, cocoamidopropyl hydroxysultaine, and disodium laureth sulfosuccinate as illustrative, but nonlimiting examples.

After uniform consistency is again achieved and the batch is allowed to stand for a few minutes, the second step is to mix propylene glycol with panthenol, which is then added to the batch in the vessel while mixing. The propylene glycol is added as a solvent to lower the freezing point of the cosmetic composition, e.g., body gel. The percentage of propylene glycol in solution can range from about 0.01% by weight to about 5.0% by weight with a preferred percentage of 1.0% by weight. Numerous other materials for lowering the freezing point of a chemical composition can be utilized including diethylene glycol (DEG), glycerine, polysorbate-20, and polysorbate-80 as illustrative, but nonlimiting, examples. The panthenol is added as an optional ingredient and functions as a conditioner for hair and skin. Panthenol, otherwise known as Pro-Vitamin B5, penetrates the cells to moisturize in order to make the hair or skin more supple. The percentage of panthenol in solution can range from about 0.01% by weight to about 5.0% by weight with a preferred percentage of 0.2% by weight of the cosmetic composition. Numerous other materials for conditioning skin and hair can be utilized including centrimonium chloride, glycereth-26, polyquaternium-7, polyquaternium-10, amodimethicone, cyclomethicone, dimethicone, dimethicone copolyol, phenyl trimethicone, stearamidopropyl dimethlamine, quaternium 15 and quaternium 18 as illustrative, but nonlimiting, examples.

The penultimate step in the process is to add a number of the optional ingredients to the batch, while mixing. The first of these ingredients includes a fragrance. The number of fragrances are innumerable with one illustrative, but nonlimiting example being FREE SPIRIT™ BELLEAIRE™. The percentage of fragrance in solution can be unlimited with a preferred range from about 0.01% by weight to about 5.0% by weight with a most preferred percentage of 0.5% by weight of the cosmetic composition.

The second of these optional ingredients includes a moisturizer or humectant. Preferably, glycereth-26 can also be added as an optional ingredient and is a vegetable-based ingredient that attracts and retains moisture to help restore a healthy balance in the hair and skin. The percentage of glycereth-26 in solution can range from about 0.01% by weight to about 5.0% by weight with a preferred percentage of 1.0% by weight. Numerous other materials for moisturizing skin and hair can be utilized including centrimonium chloride, panthanol, polyquaternium-7, polyquaternium-10, amodimethicone, cyclomethicone, dimethicone, dimethicone copolyol, phenyl trimethicone, stearamidopropyl dimethlamine, quaternium 15 and quaternium 18 as illustrative, but nonlimiting, examples.

Finally, the last of these optional ingredients includes a preservative. The preferred preservative is dimethylol dimethyl hydantoin (DMDMH). These ingredients are mixed in until the mixture is clear and uniform and then the mixing is reduced. The percentage of dimethylol dimethyl hydantoin (DMDMH) in solution can range from about 0.01% by weight to about 2.0% by weight with a preferred percentage of 0.4% by weight of the cosmetic composition. Numerous other materials that operate as a preservative can be utilized including methylparaben, propylparaben, methylchloroisothiazolinone and methylisothiazolinone as illustrative, but nonlimiting, examples.

The final step in the formulation process is to reduce mixing and carefully add the beads. In this case, the preferred form of the bead includes a shell material that houses a core oil. The possible shell materials include agar, gelatin, gelatin coacervate, methyolphenols, and polyoxymethylene urea. Available core oils can include both natural and synthetic oils such as aloe vera, avocado, boretsh, frankincense, grape seed, jojoba, lavender, macadamia nut, meadowfoam, rose hip, rosemary, safflower, seaweed, sesame, soybean, sunflower, sweet almond oil, tea tree, caprylic/capric triglyceride, cyclomethicone, isopropyl myristate, mineral oil, mineral oil substitute, polydimethyl siloxane, silicone oil, tridecyl trimellitate and neopentylglycol dicaprylate/dicapate. Other core oils include vitamins such as Vitamin A (Retinyl Palmitate and Retinol), Vitamin B5 (Panthanol Triacetate), Vitamin C (ascorbic acid) and Vitamin E (Topopherol and Acetate). Moreover, core oils can include benzophenone, biobantical extracts, copper chlorophyll, menthol, and methylsalicylate. Moreover, a wide variety of fragrances and flavors in addition to full gamut of solids and other compounds can be utilized. The percentage of beads in solution can range from about 0.01% by weight to about 2.0% by weight with a preferred percentage of 1.5% by weight of the cosmetic composition. The preferred beads are liposomes. One illustrative, but nonlimiting supplier of liposomes is Lipo Technologies, Inc., having a place of business at 800 Scholz Drive, Vandalia, Ohio 45377.

The beads are mixed until evenly dispersed. The cosmetic composition should then remain stationary until the air dissipates from within the cosmetic composition. This is a cold process where the overall pH is between 6.5 to 7.75.

The following examples are offered to illustrate aspects of the present invention, and are not intended to limit or define the present invention in any manner:

### EXAMPLE 1

A carbomer pre-mix is created by mixing 2.0 wt. % carbomer ETD 2020™ in an aqueous solution containing 97.3 wt. % water. In order to neutralize the pH, 0.7 wt. % triethanolamine (TEA) is added as a neutralizing agent and then the carbomer pre-mix is set aside. This is followed by creating the gum pre-mix, which includes 1.5 wt. % hydroxymethylcellulose in an aqueous solution. This hydroxymethylcellulose is sprinkled into the water with heavy agitation to form a thick, clear gel. After creating both the carbomer pre-mix and the gum pre-mix, the next step includes mixing surfactants, 20.0 wt. % sodium lauryl sulfate and 5.0 wt. % cocamide DEA, in a vessel and then adding the previously described carbomer pre-mix. When the solution achieves a uniform consistency, the previously described gum pre-mix is added until uniformity of the mixture is achieved and then the batch is left standing. After this uniform consistency is achieved and the batch is allowed to stand for a few minutes, the second step is to mix 1.0 wt. % glycerine with 0.5 wt. % polyquaternium-7, which is then added to the batch in the vessel while mixing. The glycerine is added as a solvent to lower the freezing point of the cosmetic composition, e.g., body gel. The polyquaternium-7 is added as an optional ingredient and functions as a cellulose polymer used as an anti-static conditioning agent. Subsequently, 0.4 wt. % dimethylol dimethyl hydantoin (DMDMH) is added as a preservative with a sufficient quantity of water and then mixing until the cosmetic composition is clear and uniform. Finally, the beads were carefully added into the mixture and mixed until evenly dispersed. In this case, the preferred form of the beads includes shell materials that house core oils of 1.5 wt. % vitamin E acetate (tocopheryl acetate).

### EXAMPLE 2

A carbomer pre-mix is created by mixing 2.0% SF-1 (AQUA™) in an aqueous solution containing 97.5 wt. % water. In order to neutralize the pH, 0.5 wt. % triethanolamine (TEA) is added as a neutralizing agent and then the carbomer premix is set aside. This is followed by creating the gum pre-mix, which includes 0.7 wt. % carboxymethylcellulose in an aqueous solution. This carboxymethylcellulose is sprinkled into the water with heavy agitation to form a thick, clear gel. After creating both the carbomer pre-mix and the gum pre-mix, the next step includes mixing a surfactant, 25.0 wt. % triethanolamine (TEA) lauryl sulfate, in a vessel and then adding the previously described carbomer pre-mix. When the solution achieves a uniform consistency, the previously described gum pre-mix is added until uniformity of the mixture is achieved and then the batch is left standing. After this uniform consistency is achieved and the batch is allowed to stand for a few minutes, the second step is to mix 1.0 wt. % propylene glycol with 0.25 wt. % panthenol, which is then added to the batch in the vessel while mixing. The propylene glycol is added as a solvent to lower the freezing point of the cosmetic composition, e.g., body gel. The panthenol is added as an optional ingredient and functions as a conditioner for hair and skin. Panthenol, otherwise known as Pro-Vitamin B5, penetrates the cells to moisturize in order to make the hair or skin more supple. Subsequently, 0.4 wt. % dimethylol dimethyl hydantoin (DMDMH) is added as a preservative along with 1.0 wt. % glycereth-26 as an optional ingredient. Glycereth-26 is a vegetable based ingredient that attracts and retains moisture to help restore a healthy balance in the hair and skin. These ingredients are mixed in, with a sufficient quantity of water, until the mixture is clear and uniform and then the mixing is reduced. Finally, the beads were carefully added into the mixture and mixed until evenly dispersed. In this case, the preferred form of the beads includes shell materials that house core oils of 1.5 wt. % by weight of vitamin A palmitate (retinyl palmitate).

### EXAMPLE 3

A carbomer pre-mix is created by mixing 1.0 wt. % SF-1 (AQUA™) in an aqueous solution containing 98.5 wt. % water. In order to neutralize the pH, 0.5 wt. % tetrahydroxypropyl ethylenediamine (TE) is added as a neutralizing agent and then the carbomer pre-mix is set aside. This is followed by creating the gum premix, which includes 1.0 wt. % hydroxyethylcellulose in an aqueous solution. This hydroxyethylcellulose is sprinkled into the water with heavy agitation to form a thick, clear gel. After creating both the carbomer pre-mix and the gum pre-mix, the next step includes mixing in surfactants, 20.0 wt. % decyl polyglucoside and 5.0 wt. % cocamide MEA, in a vessel and then adding the previously described carbomer pre-mix. When the solution achieves a uniform consistency, the previously described gum pre-mix is added until uniformity in the mixture is again achieved and then the batch is left standing. After this uniform consistency is achieved and the batch is allowed to stand for a few minutes, the second step is to mix 1.5 wt. % propylene glycol with 0.25 wt. % panthenol, which is then added to the batch in the vessel while mixing. The propylene glycol is added as a solvent to lower the freezing point of the cosmetic composition, e.g., body gel. The panthenol is added as an optional ingredient and functions as a conditioner for hair and skin. Panthenol, otherwise known as Pro-Vitamin B5, penetrates the cells to moisturize in order to make the hair or skin more supple. Subsequently, 0.2 wt. % methylchloroisothiazolinone and methylisothiazolinone is added as a preservative along with 10.5 wt. % Glycereth-26 with a sufficient quantity of water and mixed until the cosmetic composition is clear and uniform. Glycereth-26 is also an optional vegetable-based ingredient that attracts and retains moisture to help restore a healthy balance in the hair and skin. Finally, the beads were carefully added into the mixture and mixed evenly until dispersed. In this case, the preferred form of the beads include shell materials that house core oils of 1.5 wt. % aloe vera.

### EXAMPLE 4

A carbomer pre-mix is created by mixing 1.5 wt. % carbomer ETD 2020™ in an aqueous solution containing 98.15 wt. % water. In order to neutralize the pH, 0.35 wt. % sodium hydroxide is added as a neutralizing agent and then the carbomer pre-mix is set aside. This is followed by creating the gum pre-mix, which includes 1.0 wt. % carboxymethylcellulose in an aqueous solution of 99.0 wt. % water. This carboxymethylcellulose is sprinkled into the water with heavy agitation to form a thick, clear gel. After creating both the carbomer pre-mix and the gum pre-mix, the next step includes mixing in surfactants, 3.0 wt. % triethanolamine (TEA) lauryl sulfate, 18.0 wt. % sodium lauryl sulfate and 5.0 wt. % cocamide MEA, in a vessel and then adding the previously described carbomer pre-mix. When the solution achieves a uniform consistency, the previously described gum pre-mix is added until uniformity in the mixture is achieved and then the batch is left standing. After this uniform consistency is achieved and the batch is allowed to stand for a few minutes, the second step is to mix 0.5 wt. % glycerine with 0.3 wt. % polyquaternium-7, which is then added to the batch in the vessel while mixing. The glycerine is added as a solvent to lower the freezing point of the cosmetic composition, e.g., body gel. The polyquaternium-7 is added as an optional ingredient and functions as a cellulose polymer used as an anti-static conditioning agent. Subsequently, 0.4 wt. % dimethylol dimethyl hydantoin (DMDMH) is added as a preservative with a sufficient quantity of water and then mixed until the cosmetic composition is clear and uniform. Finally, the beads were carefully added into the mixture and mixed until evenly dispersed. In this case, the preferred form of the beads includes shell materials made of agar that houses core oils of 1.5 wt. % rosemary.

### EXAMPLE 5

A carbomer pre-mix is created by mixing 1.5 wt. % carbomer ETD 2020™ in an aqueous solution containing 98.15 wt. % water. In order to neutralize the pH, 0.35 wt. % sodium hydroxide is added as a neutralizing agent and then the carbomer pre-mix is set aside. This is followed by creating the gum pre-mix, which includes 25.0 wt. % carboxymethylcellulose in an aqueous solution of 75.0 wt. % water. This carboxymethylcellulose is sprinkled into the water with heavy agitation to form a thick, clear gel. After creating both the carbomer pre-mix and the gum pre-mix, the next step includes mixing in surfactants, 3.0 wt. % triethanolamine (TEA) lauryl sulfate, 18.0 wt. % sodium lauryl sulfate and 5.0 wt. % cocamide MEA, in a vessel and then adding the previously described carbomer pre-mix. When the solution achieves a uniform consistency, the previously described gum pre-mix is added until uniformity in the mixture is achieved and then the batch is left standing. After this uniform consistency is achieved and the batch is allowed to stand for a few minutes, the second step is to mix 0.5 wt. % glycerine with 0.3 wt. % polyquaternium-7, which is then added to the batch in the vessel while mixing. The glycerine is added as a solvent to lower the freezing point of the cosmetic composition, e.g., body gel. The polyquaternium-7 is added as an optional ingredient and functions as a cellulose polymer used as an anti-static conditioning agent. Subsequently, 0.4 wt. % dimethylol dimethyl hydantoin (DMDMH) is added as a preservative. These ingredients are mixed in with a sufficient quantity of water and then the mixing is reduced. Finally, the beads were carefully added into the mixture. The entire cosmetic composition had a creamy aspect that destroyed the aesthetic qualities and was entirely too viscous to perform the desired function of a body gel.

### EXAMPLE 6

A carbomer pre-mix is created by mixing 4.66 wt. % carbomer SF-1 AQUA™ in an aqueous solution containing 94.84 wt. % water. In order to neutralize the pH, 0.5 wt. % triethanolamine (TEA) is added as a neutralizing agent and then the carbomer pre-mix is set aside. This is followed by creating the gum pre-mix, which includes 2.0 wt. % carboxymethylcellulose in an aqueous solution of 98.0 wt. % water. This carboxymethylcellulose is sprinkled into the water with heavy agitation to form a thick, clear gel. After creating both the carbomer pre-mix and the gum pre-mix, the next step includes mixing in surfactant, i.e. 25.0 wt. % triethanolamine (TEA) lauryl sulfate in a vessel and then adding the previously described carbomer pre-mix. When the solution achieves a uniform consistency, the previously described gum pre-mix is added until uniformity in the mixture is achieved and then the batch is left standing. Other ingredients are then prepared for mixing in the batch. Panthenol in an amount of 0.25% by weight is mixed with 1.0% by weight of propylene glycol until it is clear and then it is added to the batch. Fragrance in an amount of 0.7% by weight and Glycereth-26 in an amount of 1.0% by weight are added. DMDM Hydantoin in an amount of 0.40% by weight is added as well. The batch is mixed until it is clear and uniform. While the mixer is off 0.25% by weight each of red, white and blue liposomes are then are added after any liquid preservative is drained off. The mixer is turned on and off until the particles are evenly dispersed in the batch. If desired a paddle may be used to assist in dispersing the liposomes by poking them down into the vortex of the mixer. This process provides a cosmetic composition having 35.2% by weight of the carbomer solution, 35.2% by weight of the gum formulation and 25% by weight of the surfactant.

Having described the invention in detail, those skilled in the art will appreciate that modifications may be made to the invention without departing from its spirit and scope. Therefore, it is not intended that the scope of the invention be limited to the specific embodiments described. Rather, it is intended that the appended claims and their equivalents determine the scope of the invention.

1. A process for suspending beads in a cosmetic formulation which comprises:
mixing a carbomer in a range from about 0.01% by weight to about 10.0% by weight with a neutralizing agent in a range from about 0.01% by weight to about 2.0% by weight to create a carbomer solution;
mixing a gum-like substance in a range from about 0.01% by weight to about 10.0% by weight to create a gum solution;
mixing the carbomer solution with the gum solution to form a cosmetic composition; and
adding at least one bead, which includes a shell that encloses a core oil, to the cosmetic composition.

2. The process according to claim 1 wherein the neutralizing agent is selected from the group consisting of triethanolamine, sodium hydroxide, tetrahydroxypropyl dimethylamine and diisopropylamine.

3. The process according to claim 1 wherein the gum-like substance is selected from the group consisting of carboxymethylcellulose, hydroxymethylcellulose, hydroxyethylcellulose, magnesium aluminum silicate, xanthan gum and guar gum.

4. The process according to claim 1 which further includes the step of adding a surfactant in an amount of about 1.0% by weight of the cosmetic formulation to about 75.0% by weight of the cosmetic formulation.

5. The process according to claim 4 wherein the surfactant is selected from the group consisting of triethanolamine lauryl sulfate, sodium lauryl sulfate, sodium laureth sulfate, ammonium lauryl sulfate, ammonium laureth sulfate, sodium C14-16 olefin sulfonate, sodium xylene sulfonate, decyl polyglucoside, cocamide DEA, cocamide MEA, lauramide DEA, lauramide MEA, sodium cocoamphodiacetate, cocoamidopropyl betaine, cocoamide oxide, cocoamidopropyl hydroxysultaine and disodium laureth sulfosuccinate.

6. The process according to claim 4 wherein the surfactant is triethanolamine lauryl sulfate.

7. The process according to claim 1 which further includes the steps of adding one or more ingredients selected from the group consisting of a chemical to lower the freezing point, a preservative and a fragrance.

8. The process according to claim 7 wherein the chemical to lower the freezing point is either glycerine or propylene glycol and the preservative is dimethylol dimethyl hydantoin.

9. The process according to claim 1 wherein the at least one bead includes a plurality of beads added in an amount from about 0.01% by weight of the cosmetic formulation to about 2.0% of the cosmetic formulation.

10. The process according to claim 1 wherein the at least one bead includes a shell housing a core oil.

11. The process according to claim 10 wherein the shell housing is selected from the group consisting of agar, gelatin, gelatin coacervate, methyolphenols, and polyoxymethylene urea.

12. The process according to claim 10 wherein the core oil is selected from the group consisting of aloe vera, avocado, boretsh, frankincense, grape seed, jojoba, lavender, macadamia nut, meadowfoam, rose hip, rosemary, safflower, seaweed, sesame, soybean, sunflower, sweet almond oil, tea tree, caprylic/capric triglyceride, cyclomethicone, isopropyl myristate, mineral oil, mineral oil substitute, polydimethyl siloxane, silicone oil, tridecyl trimellitate and neopentylglycol dicaprylate/dicapate, vitamins, benzophenone, biobantical extracts, copper chlorophyll, menthol, and methylsalicylate.

13. A process for suspending beads in a cosmetic formulation, which comprises:
mixing a carbomer in a range from about 0.01% by weight to about 10.0% by weight with a neutralizing agent in a range from about 0.01% by weight to about 2.0% by weight to create a carbomer solution having a substantially uniform consistency;
mixing a gum-like substance in a range from about 0.01% by weight to about 10.0% by weight to create a gum solution;
mixing the carbomer solution with the gum solution until a cosmetic composition having a substantially uniform consistency is achieved; and
adding to the cosmetic composition at least one bead having a shell that encloses a core oil.

14. A process according to claim 1 wherein the amount of said carbomer solution formed is from about 10-90% by weight of said cosmetic formulation, and the amount of said gum solution formed is from about 10-90% by weight of said cosmetic formulation.

15. A process according to claim 1 wherein the amount of said carbomer solution formed is from about 15-40% by weight of said cosmetic formulation, and the amount of said gum solution formed is from about 15-40% by weight of said cosmetic formulation.

16. A process according to claim 1 wherein the amount of said carbomer solution formed is from about 30-40% by weight of said cosmetic formulation, and the amount of said gum solution formed is from about 30-40% by weight of said cosmetic formulation.

17. A process according to claim 4 wherein the amount of said surfactant present is from about 15-40% by weight of said cosmetic formulation.

18. A process according to claim 4 wherein the amount of said surfactant present is from about 20-30% by weight of said cosmetic formulation.

19. A process according to claim 1 wherein the amount of said carbomer solution formed is 35.2% by weight of said cosmetic formulation, and the amount of said gum solution formed is 35.2% by weight of said cosmetic formulation.

20. A process according to claim 4 wherein the amount of said surfactant present is 25% by weight of said cosmetic formulation.

21. A process according to claim 1 wherein the amount of the at least one bead includes a plurality of beads present that is 1.5% by weight of said cosmetic formulation.

22. A cosmetic formulation which comprises:
(a) a carbomer solution which comprises a carbomer in a range from about a 0.01% by weight to about 10.0% by weight of the carbomer solution and a neutralizing agent in a range from about 0.01% by weight to about 2.0% by weight of the carbomer solution;
(b) a gum solution which comprises a gum-like substance in a range from about 0.01% by weight to about 10.0% by weight of the gum solution; and
(c) at least one bead having a shell that encloses a core oil.

23. The cosmetic formulation according to claim 22 wherein the neutralizing agent is selected from the group consisting of triethanolamine, sodium hydroxide, tetrahydroxypropyl dimethylamine and diisopropylamine.

24. The cosmetic formulation according to claim 22 wherein the gum-like substance is selected from the group consisting of carboxymethylcellulose, hydroxymethylcellulose, hydroxyethylcellulose, magnesium aluminum silicate, xanthan gum and guar gum.

25. The cosmetic formulation according to claim 22 which further includes a surfactant in an amount of about 1.0% by weight to about 75.0% by weight of the cosmetic formulation.

26. The cosmetic formulation according to claim 25 wherein the surfactant is selected from the group consisting of triethanolamine lauryl sulfate, sodium lauryl sulfate, sodium laureth sulfate, ammonium lauryl sulfate, ammonium laureth sulfate, sodium C14-16™ olefin sulfonate, sodium xylene sulfonate, decyl polyglucoside, cocamide DEA, cocamide MEA, lauramide DEA, lauramide MEA, sodium cocoamphodiacetate, cocoamidopropyl betaine, cocoamide oxide, cocoamidopropyl hydroxysultaine and disodium laureth sulfosuccinate.

27. The cosmetic formulation according to claim 25 wherein the surfactant is triethanolamine lauryl sulfate.

28. The cosmetic formulation according to claim 22 which further includes one or more ingredients selected from the group consisting of a chemical to lower the freezing point, a preservative and a fragrance.

29. The chemical formulation according to claim 28 wherein the chemical to lower the freezing point is either glycerine or propylene glycol and the preservative is dimethylol dimethyl hydantoin.

30. The cosmetic formulation according to claim 22 wherein the at least one bead includes a plurality of beads present in an amount from about 0.01% by weight to about 2.0% of the cosmetic formulation.

31. The cosmetic formulation according to claim 22 wherein the shell is selected from the group consisting of agar, gelatin, gelatin coacervate, methyolphenols, and polyoxymethylene urea.

32. The cosmetic formulation according to claim 22 wherein the core oil is selected from the group consisting of aloe vera, avocado, boretsh, frankincense, grape seed, jojoba, lavender, macadamia nut, meadowfoam, rose hip, rosemary, safflower, seaweed, sesame, soybean, sunflower, sweet almond oil, tea tree, caprylic/capric triglyceride, cyclomethicone, isopropyl myristate, mineral oil, mineral oil substitute, polydimethyl siloxane, silicone oil, tridecyl trimellitate and neopentylglycol dicaprylate/dicapate, vitamins, benzophenone, biobantical extracts, copper chlorophyll, menthol, and methylsalicylate.

33. A substantially uniformly consistent cosmetic formulation, which comprises:
(a) a substantially uniformly consistent carbomer solution comprising a carbomer in a range from about 0.01% by weight to about 10.0% by weight of said carbomer solution and a neutralizing agent in a range from about a 0.01% by weight to about 2.0% by weight of said carbomer solution;
(b) a gum solution comprising a gum-like substance in a range from about 0.01% by weight to about 10.0% by weight of said gum solution; and
(c) at least one bead having a shell that encloses a core oil.

34. A cosmetic formulation according to claim 22 wherein the amount of said carbomer solution is from about 10-90% by weight of said cosmetic formulation, and the amount of said gum solution is from about 10-90% by weight of said cosmetic formulation.

35. A cosmetic formulation according to claim 22 wherein the amount of said carbomer solution is from about 15-40% by weight of said cosmetic formulation, and the amount of said gum solution is from about 15-40% by weight of said cosmetic formulation.

36. A cosmetic formulation according to claim 22 wherein the amount of said carbomer solution is from about 30-40% by weight of said cosmetic formulation, and the amount of said gum solution is from about 30-40% by weight of said cosmetic formulation.

37. A cosmetic formulation according to claim 25 wherein the amount of said surfactant present is from about 15-40% by weight of said cosmetic formulation.

38. A cosmetic formulation according to claim 25 wherein the amount of said surfactant present is from about 20-30% by weight of said cosmetic formulation.

39. A cosmetic formulation according to claim 22 wherein the amount of said carbomer solution is 35.2% by weight of said cosmetic formulation and the amount of said gum solution is 35.2% by weight of said cosmetic formulation.

40. A cosmetic formulation according to claim 25 wherein the amount of said surfactant present is 25% by weight of said cosmetic formulation.

41. A cosmetic formulation according to claim 33 wherein the amount of the at least one bead includes a plurality of beads present is 1.5% by weight of said cosmetic formulation.
